SEQUENCE LISTING (2 sequences)

SEQ ID NO: 1 - DNA, 22 bp, synthetic construct
  source 1..22: mol_type = other DNA
ccttatcatttagaggaaggag

SEQ ID NO: 2 - DNA, 20 bp, synthetic construct
  source 1..20: mol_type = other DNA
tcctccgcttattgatatgc

(19) United States
(12) Patent Application Publication
(10) Pub. No.: US 2026/0347951 A1
(43) Pub. Date: Nov. 5, 2026

(54) METHOD FOR QUICKLY IDENTIFYING RUBUS CHINGII

(71) Applicant: ZHEJIANG PHARMACEUTICAL UNIVERSITY, Ningbo (CN)

(72) Inventor: Lixia Yuan, Ningbo (CN)

(21) Appl. No.: 19/098,393

(22) Filed: Apr. 2, 2025

(30) Foreign Application Priority Data
Mar. 25, 2025 (CN) 202510356480.3

(51) Int. Cl. C12Q 1/6895

(52) U.S. Cl. CPC C12Q 1/6895; C12Q 2600/156; C12Q 2600/16

(57) ABSTRACT

A method for quickly identifying Rubus chingii comprises collecting fruits and stem-leaf samples of Rubus chingii for preservation; extracting DNA from the original samples and preparing it as a test solution at a standard concentration; using amplification primers to amplify the test solution; Rubus chingii pseudo-samples are obtained through DNA extraction, primer amplification, and high-throughput sequencing; design of Rubus chingii multiplex ligation-dependent probe amplification probes; based on the Rubus chingii multiplex ligation-dependent probe amplification probes, the Rubus chingii DNA samples and the Rubus chingii pseudo-samples are tested by combining a melting curve, resulting in identification and detection results. The method of the invention combines Rubus chingii multiplex ligation-dependent probe amplification with the melting curve to establish a method for quickly identifying Rubus chingii adulterants.

The material in the XML file submitted for this application is incorporated herein by reference in its entirety: a total of 1 file named “sequence-listing-SFUT327”, size 2,509 bytes, created on Mar. 27, 2026.

### 1. TECHNICAL FIELD

The invention relates to the technical field of Rubus chingii identification, in particular to a method for quickly identifying Rubus chingii.

### 2. BACKGROUND ART

Traditional identification methods for Chinese medicinal materials mostly rely on morphological, microscopic, and physicochemical characteristics. However, closely related plant species often have similar external appearances, and after rough processing at the place of origin and preparation into decoction pieces, it becomes difficult to distinguish their original shape and color. Additionally, microscopic features are often damaged to varying degrees, making identification challenging. Furthermore, the chemical composition of the same medicinal material can vary significantly due to factors such as growth cycle, storage conditions, processing methods, and growing environment. As a result, relying solely on morphological and chemical characteristic analysis is no longer sufficient for accurate identification of certain plant materials. Currently, the identification of Rubus chingii faces these issues, which makes its identification quite challenging.

In recent years, with the development of molecular biology techniques, DNA fingerprint detection technology has been employed for the identification of traditional Chinese medicines. This technology directly utilizes variations in characteristic DNA sequences for species identification, unaffected by growth and development stages, tested parts, or environmental conditions; it exhibits high accuracy, and after optimization, the experimental process becomes standardized, featuring simple and rapid operation with minimal sample usage. Molecular biological identification techniques, in particular, boast unique accuracy and high genetic stability. However, the application of molecular identification techniques for detecting adulterants in Rubus chingii is relatively limited. For instance, Jie Fang et al. randomly tested 24 samples of commercially available Rubus chingii by using DNA barcoding, while Qundan Lv et al. successfully identified the original plant of Rubi Radix et Rhizoma and nine of its confusingly similar congeners using the ITS2 sequence. Researchers from our team have also previously established a PCR-RFLP (Polymerase Chain Reaction-Restriction Fragment Length Polymorphism) identification technique for Rubus chingii and its adulterants. However, these methods have certain limitations in terms of detection speed and accuracy. For example, in previous experiments conducted by our team, the PCR-RFLP identification method based on the ITS2 sequence could produce false positive results if incomplete enzyme digestion occurred, mistakenly indicating the presence of genuine Rubus chingii samples. Therefore, the development of an identification technique with high efficiency, precision, and ease of operation is urgently needed.

Multiplex Ligation-dependent Probe Amplification (MLPA) is a nucleic acid detection technology that simultaneously performs qualitative and semi-quantitative analysis of 50 different target genes in a single reaction tube; it utilizes MLPA probes to hybridize with target sequences, followed by probe-specific ligation by using a ligase enzyme, uses a pair of universal primers for PCR amplification, separates and analyzes by using capillary electrophoresis. This technique is characterized by high throughput, high specificity, fast detection speed, and ease of operation, and has been applied in areas such as detection of pathogenic microorganisms, processed food monitoring, tumor and genetic disease diagnosis, and the identification of authenticity of Chinese medicinal materials. However, traditional MLPA product analysis uses capillary electrophoresis, which is time-consuming and carries the risk of aerosol contamination during the extraction of PCR products.

Based on this, providing a method for quickly identifying Rubus chingii by using MLPA has significant practical implications.

### 3. SUMMARY OF THE INVENTION

The object of the invention is to provide a method for quickly identifying Rubus chingii, aiming to solve the technical problems in the prior art, such as the time-consuming nature of MLPA detection, as well as the low accuracy and efficiency of Rubus chingii identification methods.

In order to realize the above object, the invention is realized by the following technical scheme:
a method for quickly identifying Rubus chingii, comprising the following steps:
preparation of original samples: collecting fruits and stem-leaf samples of Rubus chingii for preservation;
DNA extraction from Rubus chingii samples: extracting DNA from the original samples and preparing it as a test solution at a standard concentration;
amplification of internal transcribed spacer (ITS) sequences of Rubus chingii: using amplification primers to amplify the test solution.
amplification of the internal transcribed spacer sequence of Rubus chingii pseudo-samples: Rubus chingii pseudo-samples are obtained through DNA extraction, primer amplification, and high-throughput sequencing;
design of Rubus chingii multiplex ligation-dependent probe amplification (MLPA) probes;
test detection: based on the Rubus chingii multiplex ligation-dependent probe amplification probes, the Rubus chingii DNA samples and the Rubus chingii pseudo-samples are tested by combining a melting curve, resulting in identification and detection results.

Further, the DNA extraction from Rubus chingii samples specifically involves using a modified CTAB (Cetyl Trimethyl Ammonium Bromide) method for extracting plant genomic DNA.

Further, the concentration of the test solution is 50-100 ng/μL.

Further, the storage condition for the test solution is at −20 °C.

Further, the amplification primers used are the universal amplification primers ITS 5afwd/4re.

Further, the test detection process includes DNA denaturation, hybridization reaction, ligation reaction, real-time fluorescent amplification, and high-resolution melting.

Further, the DNA denaturation, hybridization reaction, and ligation reaction are completed in a PCR (Polymerase chain reaction) amplifier.

Further, the real-time fluorescent amplification and high-resolution melting are conducted in an Applied Biosystems™ QuantStudio™ 3 Real-Time PCR System.

Further, after the high-resolution melting, signals are collected between 45 °C and 97 °C, a characteristic melting curve profile is analyzed by using Applied Biosystems™ QuantStudio™ 3 software to analyze the test detection results.

The invention also provides an application of the method for quickly identifying Rubus chingii of this scheme in Rubus chingii identification.

Compared with the prior art, the technical scheme of the invention has the following advantageous effects:
this invention introduces melting curve analysis, combining MLPA with High Resolution Melting (“MLPA-HRM”) on a molecular biology basis to establish a method for quickly identifying Rubus chingii and their adulterants. The melting curve diagram generated by “MLPA-HRM” are clearly visible, addressing the shortcomings of DNA barcoding, which involves complex operations, time-consuming processes, and inability to accurately identify some adulterated samples. “MLPA-HRM” also overcomes the deficiencies of PCR-RFLP, such as unclear gel electrophoresis bands and false positive results. Furthermore, “MLPA-HRM” demonstrates its advantages over traditional methods by being more informative and sensitive. Additionally, since “MLPA-HRM” is based on the utilization of ITS sequences, this technology can be applied throughout the entire production cycle of Rubus chingii medicinal materials, with a wider range of applications.

### 4. BRIEF DESCRIPTION OF ACCOMPANYING DRAWINGS

FIG. 1 is a schematic diagram of the “MLPA-HRM” reaction steps in Embodiment 1 of the invention;

FIG. 2 is a characteristic melting curve diagram of the product obtained by the “MLPA-HRM” reaction in Embodiment 1 of the invention.

### 5. SPECIFIC EMBODIMENT OF THE INVENTION

The invention provides a method for quickly identifying Rubus chingii, comprising the following steps:
preparation of original samples: collecting fruits and stem-leaf samples of Rubus chingii for preservation;
DNA extraction from Rubus chingii samples: extracting DNA from the original samples and preparing it as a test solution at a standard concentration;
amplification of internal transcribed spacer (ITS) sequences of Rubus chingii: using amplification primers to amplify the test solution.
amplification of the internal transcribed spacer sequence of Rubus chingii pseudo-samples: Rubus chingii pseudo-samples are obtained through DNA extraction, primer amplification, and high-throughput sequencing;
design of Rubus chingii multiplex ligation-dependent probe amplification (MLPA) probes;
test detection: based on the Rubus chingii multiplex ligation-dependent probe amplification probes, the Rubus chingii DNA samples and the Rubus chingii pseudo-samples are tested by combining a melting curve, resulting in identification and detection results.

Preparation of original samples: collecting fruits and stem-leaf samples of Rubus chingii for preservation;
the collection sites of the original samples are selected from five provinces: Zhejiang, Anhui, Fujian, Jiangxi, and Jiangsu. At least three sampling points are chosen in each province. For each sampling site, 2-3 plants with similar height, morphology, and habitat are selected, ensuring no obvious pests or diseases. The specific address and growth conditions of each sampling site are recorded. During sampling, fruits and stem-leaf samples of Rubus chingii are collected and preserved according to the required procedures.

DNA extraction from Rubus chingii samples: extracting DNA from the original samples and preparing it as a test solution at a standard concentration;
the method of this invention for extracting DNA templates from Rubus chingii samples employs a modified CTAB approach for extracting plant genomic DNA. Placing approximately 50 mg of Rubus chingii sample in a 2 mL Eppendorf (EP) tube, adding two small steel beads, and then grinding them into powder by using a high-throughput tissue grinder; taking an appropriate amount of the powdered sample into a 2 mL EP tube, and adding preheated CTAB solution to lyse the cells through water bath treatment; after centrifugation, collecting the supernatant; adding chloroform-isoamyl alcohol solution (24:1) and mixing well, followed by centrifugation to obtain a top layer of the three-phase mixture; adding an equal volume of ice-cold isopropanol to the supernatant, centrifuging and discarding the supernatant (alternatively, passing through a column and discarding the filtrate); washing the precipitate or column with 75% ethanol; adding an equal amount of DDH₂O (Double-distilled water) to dissolve DNA; measuring the concentration and purity of the DNA by using a Nano-drop 2000 spectrophotometer, and selecting samples with an A260/280 ratio between 1.80 and 2.0, adjusting the concentration of these samples to be consistent (50-100 ng/μL) by using DDH₂O, and they are stored in a −20 °C refrigerator for use in subsequent experiments.

In the invention, the DNA extraction from Rubus chingii samples specifically involves using a modified CTAB (Cetyl Trimethyl Ammonium Bromide) method for extracting plant genomic DNA.

In the invention, the concentration of the test solution is 50-100 ng/μL.

In the invention, the storage condition for the test solution is at −20 °C.

Amplification of internal transcribed spacer (ITS) sequences of Rubus chingii: using amplification primers to amplify the test solution.

This invention utilizes universal amplification primers ITS 5afwd/4re (CCTTATCATTTAGAGGAAGGAG/TCCTCCGCTTATTGATATGC) targeting the ITS sequence of the plant ribosomal genome to amplify the samples. The amplification system is 50 μL, consisting of 25 μL of 2×NG PCR Master Mix, 2 μL each of forward and reverse primers, 20 μL of ddH₂O, and 1 μL of total DNA. The amplification protocol is as follows: 95 °C for 5 min; (95 °C for 30 s, 55 °C for 30 s, 72 °C for 30 s) 35 cycles; 72 °C for 10 min; 4 °C holding. The amplification products are detected by 1.5% agarose gel electrophoresis. The products with a single band and the correct length are purified by using a DNA purification kit and then diluted to 100 ng/μL with Tris-EDTA (TE) buffer; these purified products are stored at −20 °C for use in subsequent experiments and high-throughput sequencing. The high-throughput sequencing samples are sent to a biotechnology company for sequencing, performing bidirectional sequencing with the same primers as used in the PCR. The sequenced sequences are then assembled and proofread by using DNAman 10.0 software. The sequencing files are manually adjusted by using MEGA-X software. Finally, the sequencing results are output in FASTA format.

In the invention, the amplification primers used are the universal amplification primers ITS 5afwd/4re.

Amplification of the internal transcribed spacer sequence of Rubus chingii pseudo-samples: Rubus chingii pseudo-samples are obtained through DNA extraction, primer amplification, and high-throughput sequencing;

The invention obtains other adulterants, such as Rubus parvifolius and Rubus hirsutus, through DNA extraction, ITS universal primer amplification, and high-throughput sequencing.

Design of Rubus chingii multiplex ligation-dependent probe amplification (MLPA) probes;
firstly, amplifying the ITS sequences by using universal primers via PCR; subsequently, obtaining the sequencing results through high-throughput sequencing, and align the sequences with those in the National Center for Biotechnology Information (NCBI) database to confirm that the amplification products are ITS sequences, organizing all obtained sequences into Fasta files to prepare for subsequent sequence comparison and SNP (single nucleotide polymorphism) site analysis.

Using MEGA software to conduct comparative analysis to identify stable and differential SNP sites among various species, selecting appropriate-length bases as hybridization sequences on both the left and right sides of the specific SNP sites, while adding a certain length of filler sequence to ensure that the specific probes for different species have different melting temperatures (Tm), facilitating their differentiation during the melting curve analysis. In addition to considering Tm differences, factors such as probe length, GC content, and secondary structure for the “MLPA-HRM” probes need to be taken into account. The length, GC %, and Tm values of the “MLPA-HRM” probes are predicted by using RAW software; the secondary structure is assessed by using the online website mfold WebServer with the following parameter settings: [Na+]=0.35M, Folding temperature=65 °C, and other parameters set to default values; the ΔG value of the detection results should be greater than 0. Then, the Tm values for each pair of complete probe segments are predicted by using RaW-Probe software; the Tm values for the segments of different species that need to be differentiated should differ by at least 2 °C to ensure that each pair of probes generates a specific melting curve peak. Finally, the probes are synthesized and HPLC (High Performance Liquid Chromatography) purified by a biotechnology company.

Test detection: based on the Rubus chingii multiplex ligation-dependent probe amplification probes, the Rubus chingii DNA samples and the Rubus chingii pseudo-samples are tested by combining a melting curve, resulting in identification and detection results.

In the invention, before performing the “MLPA-HRM” experiment, the single probe solution and mixed probe solution are prepared. Single probe solution: taking each 1.2 μL of the working solution of the Rubus chingii left and right probes, and each 0.4 μL of the working solution of the non-Rubus chingii left and right probes, adding TE buffer to make the total volume up to 600 μL. Mixed probe solution: taking each 1.2 μL of the working solution of the Rubus chingii left and right probes, and each 0.4 μL of the working solution of the non-Rubus chingii left and right probes, adding TE buffer to make the total volume up to 600 L.

The “MLPA-HRM” reaction includes DNA denaturation, hybridization reaction, ligation reaction, real-time fluorescent amplification and high-resolution melting. The first three steps are completed in the PCR amplifier, and the real-time fluorescent amplification and high-resolution melting are conducted in an Applied Biosystems™ QuantStudio™ 3 Real-Time PCR System. After the high-resolution melting of the product, signals are collected between 45 °C and 97 °C, a characteristic melting curve profile is analyzed by using Applied Biosystems™ QuantStudio™ 3 software to analyze the test detection results, and TE buffer is used instead of DNA template solution as a blank control.

In the invention, the test detection process includes DNA denaturation, hybridization reaction, ligation reaction, real-time fluorescent amplification, and high-resolution melting.

In the invention, the DNA denaturation, hybridization reaction, and ligation reaction are completed in a PCR (Polymerase chain reaction) amplifier.

In the invention, the real-time fluorescent amplification and high-resolution melting are conducted in an Applied Biosystems™ QuantStudio™ 3 Real-Time PCR System.

In the invention, after the high-resolution melting, signals are collected between 45 °C and 97 °C, a characteristic melting curve profile is analyzed by using Applied Biosystems™ QuantStudio™ 3 software to analyze the test detection results.

The invention also provides an application of the method for quickly identifying Rubus chingii of this scheme in Rubus chingii identification.

In the invention, unless otherwise specified, the raw materials required for preparation are commercially available products well known to those skilled in the art.

The technical scheme provided by the invention is described in detail below in combination with the embodiments, but they should not be construed as limiting the protection scope of the invention.

### Embodiment 1

(1) Preparation of original samples: collecting fruits and stem-leaf samples of Rubus chingii for preservation;
the collection sites of the original samples are selected from five provinces: Zhejiang, Anhui, Fujian, Jiangxi, and Jiangsu. At least three sampling points are chosen in each province. For each sampling site, 2-3 plants with similar height, morphology, and habitat are selected, ensuring no obvious pests or diseases. The specific address and growth conditions of each sampling site are recorded. During sampling, fruits and stem-leaf samples of Rubus chingii are collected and preserved according to the required procedures.
(2) DNA extraction from Rubus chingii samples: extracting DNA from the original samples and preparing it as a test solution at a standard concentration;
the method of this invention for extracting DNA templates from Rubus chingii samples employs a modified CTAB approach for extracting plant genomic DNA. Placing approximately 50 mg of Rubus chingii sample in a 2 mL Eppendorf (EP) tube, adding two small steel beads, and then grinding them into powder by using a high-throughput tissue grinder; taking an appropriate amount of the powdered sample into a 2 mL EP tube, and adding preheated CTAB solution to lyse the cells through water bath treatment; after centrifugation, collecting the supernatant; adding chloroform-isoamyl alcohol solution (24:1) and mixing well, followed by centrifugation to obtain a top layer of the three-phase mixture; adding an equal volume of ice-cold isopropanol to the supernatant, centrifuging and discarding the supernatant (alternatively, passing through a column and discarding the filtrate); washing the precipitate or column with 75% ethanol; adding an equal amount of DDH₂O (Double-distilled water) to dissolve DNA; measuring the concentration and purity of the DNA by using a Nano-drop 2000 spectrophotometer, and selecting samples with an A260/280 ratio between 1.80 and 2.0, adjusting the concentration of these samples to be consistent (50-100 ng/μL) by using DDH₂O, and they are stored in a −20 °C refrigerator for use in subsequent experiments.
(3) Amplification of internal transcribed spacer (ITS) sequences of Rubus chingii: using amplification primers to amplify the test solution. This invention utilizes universal amplification primers ITS 5afwd/4re (CCTTATCATTTAGAGGAAGGAG/TCCTCCGCTTATTGATATGC) targeting the ITS sequence of the plant ribosomal genome to amplify the samples. The amplification system is 50 μL, consisting of 25 μL of 2×NG PCR Master Mix, 2 μL each of forward and reverse primers, 20 μL of ddH₂O, and 1 μL of total DNA. The amplification protocol is as follows: 95 °C for 5 min; (95 °C for 30 s, 55 °C for 30 s, 72 °C for 30 s) 35 cycles; 72 °C for 10 min; 4 °C holding. The amplification products are detected by 1.5% agarose gel electrophoresis. The products with a single band and the correct length are purified by using a DNA purification kit and then diluted to 100 ng/μL with Tris-EDTA (TE) buffer; these purified products are stored at −20 °C for use in subsequent experiments and high-throughput sequencing. The high-throughput sequencing samples are sent to a biotechnology company for sequencing, performing bidirectional sequencing with the same primers as used in the PCR. The sequenced sequences are then assembled and proofread by using DNAman 10.0 software. The sequencing files are manually adjusted by using MEGA-X software. Finally, the sequencing results are output in FASTA format.
(4) Amplification of the internal transcribed spacer sequence of Rubus chingii pseudo-samples: Rubus chingii pseudo-samples such as Rubus parvifolius and Rubus hirsutus are obtained through DNA extraction, primer amplification, and high-throughput sequencing.
(5) Design of Rubus chingii multiplex ligation-dependent probe amplification (MLPA) probes; firstly, amplifying the ITS sequences by using universal primers via PCR; subsequently, obtaining the sequencing results through high-throughput sequencing, and align the sequences with those in the National Center for Biotechnology Information (NCBI) database to confirm that the amplification products are ITS sequences, organizing all obtained sequences into Fasta files to prepare for subsequent sequence comparison and SNP (single nucleotide polymorphism) site analysis. Using MEGA software to conduct comparative analysis to identify stable and differential SNP sites among various species, selecting appropriate-length bases as hybridization sequences on both the left and right sides of the specific SNP sites, while adding a certain length of filler sequence to ensure that the specific probes for different species have different melting temperatures (Tm), facilitating their differentiation during the melting curve analysis. In addition to considering Tm differences, factors such as probe length, GC content, and secondary structure for the “MLPA-HRM” probes need to be taken into account. The length, GC %, and Tm values of the “MLPA-HRM” probes are predicted by using RAW software; the secondary structure is assessed by using the online website mfold WebServer with the following parameter settings: [Na+]=0.35M, Folding temperature=65 °C, and other parameters set to default values; the ΔG value of the detection results should be greater than 0. Then, the Tm values for each pair of complete probe segments are predicted by using RaW-Probe software; the Tm values for the segments of different species that need to be differentiated should differ by at least 2 °C to ensure that each pair of probes generates a specific melting curve peak. Finally, the probes are synthesized and HPLC (High Performance Liquid Chromatography) purified by a biotechnology company.
(6) Test detection: based on the Rubus chingii multiplex ligation-dependent probe amplification probes, the Rubus chingii DNA samples and the Rubus chingii pseudo-samples are tested by combining a melting curve, resulting in identification and detection results. Before performing the “MLPA-HRM” experiment, the single probe solution and mixed probe solution are prepared. Single probe solution: taking each 1.2 μL of the working solution of the Rubus chingii left and right probes, and each 0.4 μL of the working solution of the non-Rubus chingii left and right probes, adding TE buffer to make the total volume up to 600 L. Mixed probe solution: taking each 1.2 μL of the working solution of the Rubus chingii left and right probes, and each 0.4 μL of the working solution of the non-Rubus chingii left and right probes, adding TE buffer to make the total volume up to 600 L.

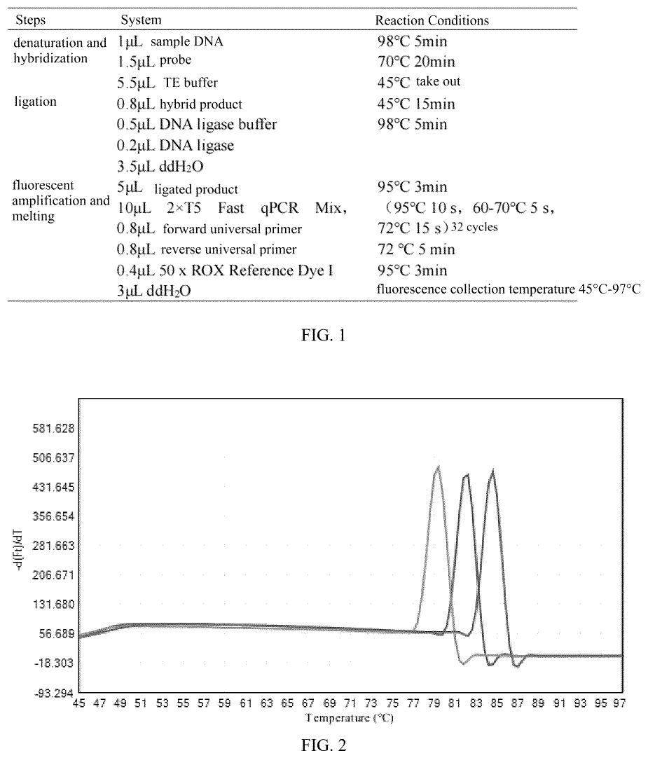

The “MLPA-HRM” reaction includes DNA denaturation, hybridization reaction, ligation reaction, real-time fluorescent amplification and high-resolution melting. The first three steps are completed in the PCR amplifier, and the real-time fluorescent amplification and high-resolution melting are conducted in an Applied Biosystems™ QuantStudio™ 3 Real-Time PCR System, the reaction steps are shown in FIG. 1.

After the high-resolution melting of the product, signals are collected between 45 °C and 97 °C, a characteristic melting curve profile is analyzed by using Applied Biosystems™ QuantStudio™ 3 software to analyze the test detection results, and TE buffer is used instead of DNA template solution as a blank control, the results are shown in FIG. 2; based on FIG. 2, it can be seen that the curve peaks from left to right are Rubus hirsutus, Rubus parvifolius and Rubus chingii.

The above method is used to detect the samples, observe their curve spectrum, and then identify Rubus chingii.

The above is only a preferred embodiment of the invention. It should be noted that, for ordinary technicians in this technical field, several improvements and modifications can be made without departing from the principle of the invention, and these improvements and modifications should also be regarded as the protection scope of the invention.

1. A method for quickly identifying Rubus chingii, comprising the following steps:
preparation of original samples: collecting fruits and stem-leaf samples of Rubus chingii for preservation;
DNA extraction from Rubus chingii samples: extracting DNA from the original samples and preparing it as a test solution at a standard concentration;
amplification of internal transcribed spacer (ITS) sequences of Rubus chingii: using amplification primers to amplify the test solution;
amplification of the internal transcribed spacer sequence of Rubus chingii pseudo-samples: Rubus chingii pseudo-samples are obtained through DNA extraction, primer amplification, and high-throughput sequencing;
design of Rubus chingii multiplex ligation-dependent probe amplification (MLPA) probes;
test detection: based on the Rubus chingii multiplex ligation-dependent probe amplification probes, the Rubus chingii DNA samples and the Rubus chingii pseudo-samples are tested by combining a melting curve, resulting in identification and detection results.

2. The method for quickly identifying Rubus chingii of claim 1, wherein the DNA extraction from Rubus chingii samples specifically involves using a modified CTAB (Cetyl Trimethyl Ammonium Bromide) method for extracting plant genomic DNA.

3. The method for quickly identifying Rubus chingii of claim 1, wherein the concentration of the test solution is 50~100 ng/μL.

4. The method for quickly identifying Rubus chingii of claim 1, wherein the storage condition for the test solution is at −20 °C.

5. The method for quickly identifying Rubus chingii of claim 1, wherein the amplification primers used are the universal amplification primers ITS 5afwd/4re.

6. The method for quickly identifying Rubus chingii of claim 1, wherein the test detection process includes DNA denaturation, hybridization reaction, ligation reaction, real-time fluorescent amplification, and high-resolution melting.

7. The method for quickly identifying Rubus chingii of claim 6, wherein the DNA denaturation, hybridization reaction, and ligation reaction are completed in a PCR (Polymerase chain reaction) amplifier.

8. The method for quickly identifying Rubus chingii of claim 6, wherein the real-time fluorescent amplification and high-resolution melting are conducted in an Applied Biosystems™ QuantStudio™ 3 Real-Time PCR System.

9. The method for quickly identifying Rubus chingii of claim 6, wherein after the high-resolution melting, signals are collected between 45 °C and 97 °C, a characteristic melting curve profile is analyzed by using Applied Biosystems™ QuantStudio™ 3 software to analyze the test detection results.

10. The method for quickly identifying Rubus chingii of claim 1, wherein this method is applied in Rubus chingii identification.
